(19) United States
(12) Patent Application Publication
(10) Pub. No.: US 2014/0121740 A1
(43) Pub. Date: May 1, 2014

(54) METHOD AND SYSTEM FOR NEUROSTIMULATION FOR THE TREATMENT OF NICOTINE ADDICTION

(71) Applicants: Lorne PATTERSON, Edgeworthstown (IE); Joseph R. WINSTON, Chapel Hill, NC (US)

(72) Inventors: Lorne PATTERSON, Edgeworthstown (IE); Joseph R. WINSTON, Chapel Hill, NC (US)

(21) Appl. No.: 14/070,033

(22) Filed: Nov. 1, 2013

Related U.S. Application Data
(60) Provisional application No. 61/721,136, filed on Nov. 1, 2012.

(51) Int. Cl. A61N 1/36

(52) U.S. Cl. CPC A61N 1/36089
USPC 607/116

(57) ABSTRACT

The present invention describes a specific set of CES waveforms and delivery schedule which significantly reduce the symptoms of acute withdrawal and protracted abstinence that typically arise following abrupt cessation of nicotine intake, and a system for delivery of same.

### CROSS-REFERENCE TO RELATED APPLICATIONS

This application is based on, and claims priority to, U.S. Provisional Application No. 61/721,136, filed Nov. 1, 2012, the entire contents of which is fully incorporated herein by reference.

### BACKGROUND OF THE INVENTION

Cranial Electrotherapy Stimulation (CES) stimulator devices are typically used for anxiety and stress, and for the treatment of addiction to opioids, cocaine, alcohol, benzodiazepines and other addictive substances. CES stimulator devices typically have user-controlled and/or processor-controlled output controls that alter the wave shape, frequency, pulse width, modulation, intensity, and/or other waveform characteristics over short (sub-second) and long (multi-day) periods of time as appropriate to the specific intervention.

### SUMMARY OF THE INVENTION

The present invention describes a specific set of CES waveforms and delivery schedule which significantly reduce the symptoms of acute withdrawal and protracted abstinence that typically arise following abrupt cessation of nicotine intake, and a system for delivery of same.

An algorithmic approach to the use of CES for addiction is described in U.S. Pat. No. 4,646,744 issued to Capel, the entire contents of which is incorporated fully herein by reference. The Capel patent describes alternating-current bi-phasic square waveforms with no net DC-component applied across the head with pulse trains having off periods of increasing duration. In particular, the Capel patent addresses Nicotine addiction as follows:

“Before a cigarette-smoker experiences a craving for a cigarette, a signal having a frequency of 5 Hz and a pulse width of 0.1 msec. is applied in an alternating current. The signal is applied intermittently in equal on and off cycles. Initially, the duration of the on and off cycles are each 5 seconds, but this duration increases in a fixed arithmetic progression by increments of one minute to a maximum duration of just over 30 minutes each, at which point the duration of the on and off periods decreases progressively in one minute increments, finally returning to the original 5 second periods. The progression is repeated four additional times over a period of 7 days.”

The present invention uses wave shapes similar to those described in the Capel patent, but uses different frequencies and pulse widths, has no off cycles, typically requires administration over only 48-72 hours, and is most effective if initiated following the onset of symptoms of acute nicotine withdrawal.

A manually controlled neurostimulator device to be used for the treatment of narcotics and alcohol is described in U.S. Pat. No. 4,865,048 issued to Eckerson, the entire contents of which is incorporated fully herein by reference. The Eckerson device delivers to the patient a train of electrical pulses comprised of square waves and spikes at 100 Hz where the square waves are 1 ms in width and the pulse trains have a duty cycle of 85% (providing off cycles, similar in concept to those of the Capel patent).

The present invention does not use waveshapes as described in the Eckerson patent, does not use pulse trains at 100 Hz or pulse widths of 1 ms, has no off cycles, and specifically addresses nicotine addiction as opposed to narcotics and alcohol.

An automated neurostimulator device to be used for alleviating pain and the symptoms of withdrawal during detoxification is described in U.S. Pat. No. 5,593,432 issued to Crowther, the entire contents of which is incorporated fully herein by reference. The Crowther patent describes a device that delivers current controlled electrical pulses comprised of generally square waves having frequencies of 90 Hz and 300 Hz and pulse widths of 100 to 300 microseconds. Algorithmic treatment programs are put forward for heroin, alcohol, chronic pain, cocaine and amphetamines, all of which incorporate off cycles.

The present invention is efficacious with either constant-voltage or constant-current output. In general, however, the use of constant current output as described in the Crowther patent is discouraged as it produces painful and abrupt changes in sensation level to the patient when pressure applied to the contact causes an impedance change or when the contact area has a sudden change in cross-sectional area. In addition, the present invention does not use pulse trains at 90 Hz or 300 Hz, specifically addresses nicotine addiction (as opposed to the addictions specified in the Crowther patent), and has no off cycles.

The present invention is efficacious when delivered by a CES device of the form described in U.S. Pat. No. 8,204,601 issued to Moyer, the entire contents of which being fully incorporated hereby by reference.

### BRIEF DESCRIPTION OF THE DRAWINGS

FIG. 1 illustrates the measurement of the rapid reduction in both acute and chronic withdrawal symptoms with a self-reported withdrawal symptom severity scale;

FIG. 2 illustrates the typical withdrawal severity scores following abrupt cessation of nicotine and the start of Neurostimulation; and

FIG. 3 illustrates a set of frequencies delivered to the patient according to the time of day.

### DETAILED DESCRIPTION

### Nicotine Cessation

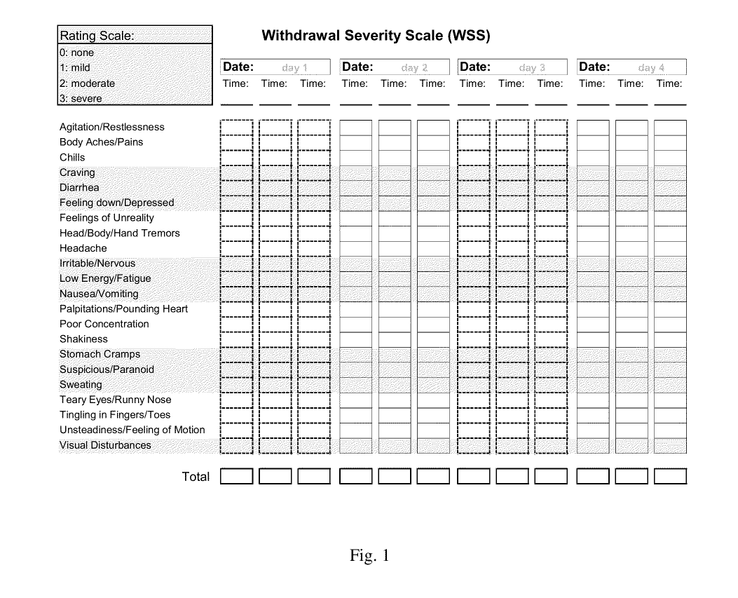

The rapid reduction in both acute and chronic withdrawal symptoms can be measured with a self-reported withdrawal symptom severity scale as shown in FIG. 1.

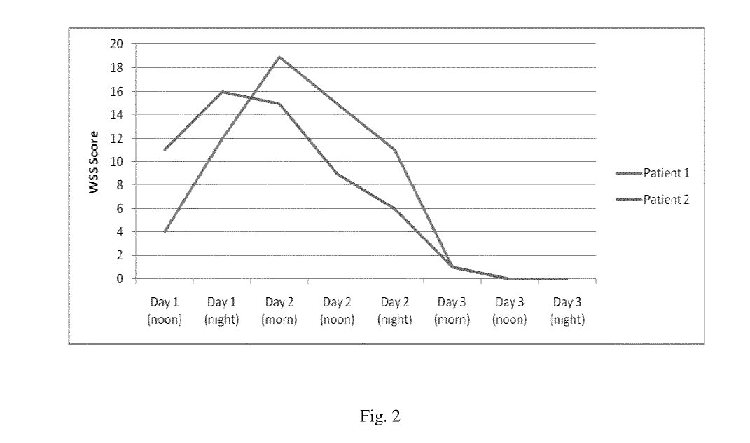

Typical withdrawal severity scores following abrupt cessation of nicotine and the start of neurostimulation look as shown in FIG. 2.

The present invention describes a specific set of CES waveforms and delivery schedule which significantly reduce the symptoms of acute withdrawal and protracted abstinence that typically arise following abrupt cessation of nicotine intake.

The neurostimulation commences following abrupt nicotine cessation and preferably after the patient has begun to exhibit acute withdrawal symptoms. During the period of stimulation, the patient should not receive any psychoactive substances.

The present invention is for stimulation applied to the head of the patient at the mastoid processes.

The duration of stimulation is typically between 48 and 72 hours. The stimulation algorithm below extends across 5 days to support individuals whose detoxification period is of atypically long duration.

### CES Waveform

The CES waveform is a an alternating current polarized bi-phasic generally square shaped pulse train with no net DC-offset. The output may be either constant current or constant voltage, with a maximum voltage typically less than 70 volts across a 10 KOhm load. Output voltage may be set by the patient at a level of comfort, typically just below or just above the level of sensation. Within this range, treatment duration and efficacy are independent of output intensity. The pulse width of the positive-going square wave is 220 μs (microseconds), with the pulse width of the negative-going portion changing with output frequency.

### Nicotine Specific Treatment Algorithm

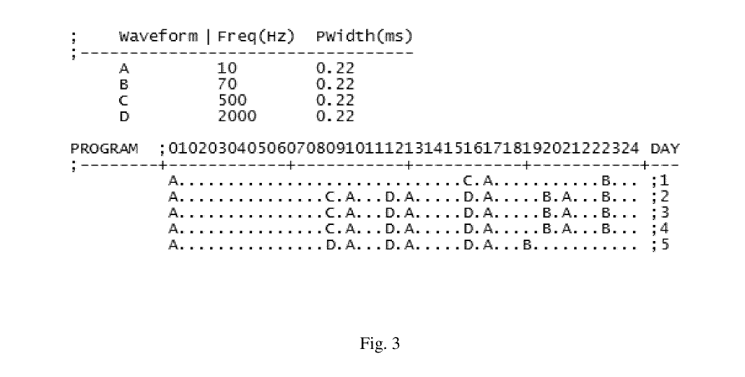

The present invention defines a set of frequencies delivered to the patient according to the time of day as shown in FIG. 3.

As shown in FIG. 3, five (5) days of treatment are defined where each treatment day starts at 1:00am, waveform changes occur on half-hour boundaries, the character “.” is used to indicate continuation of the immediately preceding waveform. The patient's first calendar day of treatment is always considered “Day 1”, independent of the time of commencement of treatment.

The above-described steps can be implemented using standard well-known programming techniques. The novelty of the above-described embodiment lies not in the specific programming techniques but in the use of the steps described to achieve the described results. Software programming code which embodies the present invention is typically stored in permanent storage. In a client/server environment, such software programming code may be stored with storage associated with a server. The software programming code may be embodied on any of a variety of known media for use with a data processing system, such as a diskette, or hard drive, or CD ROM. The code may be distributed on such media, or may be distributed to users from the memory or storage of one computer system over a network of some type to other computer systems for use by users of such other systems. The techniques and methods for embodying software program code on physical media and/or distributing software code via networks are well known and will not be further discussed herein.

It will be understood that each element of the illustrations, and combinations of elements in the illustrations, can be implemented by general and/or special purpose hardware-based systems that perform the specified functions or steps, or by combinations of general and/or special-purpose hardware and computer instructions.

These program instructions may be provided to a processor to produce a machine, such that the instructions that execute on the processor create means for implementing the functions specified in the illustrations. The computer program instructions may be executed by a processor to cause a series of operational steps to be performed by the processor to produce a computer-implemented process such that the instructions that execute on the processor provide steps for implementing the functions specified in the illustrations. Accordingly, the figures support combinations of means for performing the specified functions, combinations of steps for performing the specified functions, and program instruction means for performing the specified functions.

While there has been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

We claim:

1. A method of treating a subject for addiction to nicotine with an electrical signal transcranially comprising successive signal trains wherin
a. Said signal is continuously delivered with no “off” periods,
b. Said signal includes at least one signal train with frequency of about 10 Hz,
c. Said signal includes one or more signal trains with frequency other than 10 Hz

2. A method of claim 1, wherein one or more of the signal trains have pulse width of about 0.22 ms.

3. A method of claim 1, wherein one or more of the signal trains have frequency of about 70 Hz.

4. A method of claim 1, wherein one or more of the signal trains have frequency of about 500 Hz.

5. A method of claim 1, wherein one or more of the signal trains have frequency of about 2000 Hz.

6. A method of claim 1, wherein signal trains are delivered as per the “Nicotine specific treatment algorithm” above.

7. The method of claim 1, wherein said treatment algorithm is of 5 days duration or less.
